(19) United States
(12) Patent Application Publication
(10) Pub. No.: US 2011/0196243 A1
(43) Pub. Date: Aug. 11, 2011

(54) NON-CONTACT DETECTION OF PHYSIOLOGICAL DATA USING STOCHASTIC RESONANCE

(76) Inventors: Riheng Wu, Carthage, MO (US); Jason Turner, Joplin, MO (US); Travis Brummett, Carthage, MO (US); Caleb Browning, Carthage, MO (US)

(21) Appl. No.: 12/701,402

(22) Filed: Feb. 5, 2010

(51) Int. Cl. A61B 5/0205

(52) U.S. Cl. 600/484

(57) ABSTRACT

Obtaining physiological data from a living being using non-contact sensors is disclosed. The non-contact sensors are disposed within, for example, a vehicle seat or bed. Signals detected by the non-contacting sensors, which include both physiological signals and ambient noise, are transmitted to a processing device which is positioned nearby; such as in the vehicle seat or bed. The processing device employs a stochastic resonance function whereby a signal-to-noise ratio of the physiological signals is improved. A wavelet multi-scale decomposition function and an adaptive threshold function are then used to identify the physiological signals and distinguish them from the ambient noise. The resulting signals are measured and then made available at an output of the processing device for transmission to and use by a human-machine interface.

### BACKGROUND OF THE INVENTION

The present invention relates, generally, to systems and methods for measuring physiological signals of a living being. More particularly, the present invention relates to detecting heart rate and respiration using non-contact sensors incorporated into, for example, a vehicle seat or bed to monitor an occupant.

Systems for monitoring heart rate and respiration using contact sensors are known. Contact sensors are, in general, able to detect physiological signals quite accurately even in “noisy” environments. However, there are many instances where the placement of such sensors directly on the body of a person (or other living being) is inconvenient and/or impractical.

For example, when monitoring a person in a bed, if the person needs to move from the bed, it is necessary to either a) remove the contact sensors from the person's body, or b) transport the associated equipment to which the sensors are attached along with the person.

Another example is a system by which it is desirable to monitor the heart rate and respiration of the occupant of a vehicle. In a vehicle, it is impractical to place physiological sensors directly on the body of the occupant because unhindered movement from the vehicle seat is necessary. In addition, contact sensors may distract the occupant, which is particularly undesirable if the occupant is a vehicle driver.

In light of the above concerns, non-contact sensing systems have been developed which attempt to monitor the desired physiological data.

### BRIEF SUMMARY OF THE INVENTION

While more convenient than a contact sensor system, non-contact sensor systems are more susceptible to ambient noise as well as discrepancies in signal strength due to movement of the subject being monitored. Ambient noise is particularly evident when attempting to monitor an occupant of a vehicle since the vibrations which occur due to the operation of the vehicle itself can create electrical (and other interfering) noise. Accordingly, an improved system is needed by which physiological signals may be detected in a non-contacting manner, distinguished from ambient noise, and accurately measured.

Embodiments of the invention are directed to a method and system which combines non-contact sensors for detecting physiological signals along with a processing device which employs a stochastic resonance function to enhance the signal-to-noise ratio (SNR) of the physiological signals. Physiological signals are, in general, relatively weak, non-linear, and periodic. Ambient noise, on the other hand, is significantly stronger. Thus, the presence of ambient noise usually impedes the effective detection and measurement of the physiological signals. A stochastic resonance function, however, uses ambient noise to improve the SNR of the physiological signals.

In one embodiment, the non-contact sensors and associated processing device are incorporated into the seat of a vehicle. Preferably, the non-contact sensors are disposed both in the seat back and seat bottom of the vehicle seat so as to improve detection of heart rate and respiration. Signals detected by the non-contact sensors, along with ambient noise, are then transmitted to the associated processing device. The processing device then employs a stochastic resonance function to increase the SNR of the physiological signals. The processing device also employs a wavelet multi-scale decomposition function to further differentiate and identify the physiological signals. Thereafter, actual measurements of both the heart rate and respiration are derived.

In another embodiment, the non-contact sensors and associated processing device are incorporated into a bed such as a hospital bed. The non-contact sensors are positioned within the mattress of the bed so as to detect heart rate and respiration. Signals from the non-contact sensors are then sent to the associated processing device for further processing as described with respect to the vehicle-seat embodiment.

In one example embodiment, the non-contact sensors are piezoelectric pressure sensors for sensing vibrations. A data acquisition device is incorporated between the non-contact sensors and the processing device for performing an analog-to-digital conversion of the physiological signals and the ambient noise prior to being sent to the processing device. The processing device includes a summation module for adding the physiological signals together prior to their processing by the stochastic resonance function. The processing device also includes a data decimation module for taking the physiological signals and the ambient noise from a high sampling rate down to a lower sampling rate for further processing. In certain implementations a db5 wavelet is used as part of the wavelet multi-scale decomposition function, and the processing device includes an envelope detector for eliminating negative frequencies of the physiological signals and the ambient noise for better data measurement. The processing device also includes an adaptive threshold function for incorporating an adaptive threshold between the physiological signals and the ambient noise for better ongoing differentiation between the two.

Additional features and advantages of the present invention are described in, and will be apparent from, the following Detailed Description of the Invention and the Drawing.

### BRIEF DESCRIPTION OF THE SEVERAL VIEWS OF THE DRAWING

FIG. 1 is a schematic block diagram showing, generally, a system for the non-contact detection of physiological data;

FIG. 2 is a side view showing a system for the non-contact detection of physiological data incorporated into a vehicle seat with an occupant disposed thereon;

FIG. 3 is a front elevational view of the system and vehicle seat shown in FIG. 2 without the occupant disposed thereon;

FIG. 4 is a side view showing a system for the non-contact detection of physiological data incorporated into a bed with an occupant disposed thereon;

FIG. 5 is a top view of the system and bed shown in FIG. 4 without the occupant disposed thereon;

FIG. 6 is a schematic block diagram showing an internal structure of the Digital Signal Processor (DSP) shown in FIG. 1;

FIG. 7 is a schematic block diagram showing an internal structure of the wavelet decomposition and reconstruction module function shown in FIG. 6;

FIG. 8 is a flow chart illustrating a method for the non-contact detection of physiological data;

FIG. 9 is a waveform graph illustrating raw data of physiological signals and ambient noise sensed by non-contact pressure sensors;

FIGS. 10a-10g are waveform graphs illustrating db5 wavelet multi-scale decomposition coefficients of the physiological signals and ambient noise waveforms shown in FIG. 9;

FIGS. 11a-11f are waveform graphs illustrating db5 wavelet reconstruction of the corresponding graphs shown in FIGS. 10b-10g;

FIG. 12 is a waveform graph illustrating a first order derivative of the waveform of the d4 component shown in FIG. 11c;

FIG. 13 is a waveform graph illustrating the Hilbert Transform (HT) of the waveform shown in FIG. 12;

FIG. 14 is a waveform graph illustrating the analytic signal envelope of the waveform shown in FIG. 13; and

FIG. 15 is a waveform graph illustrating an application of an Adaptive Threshold Function (ATF) to the waveform shown in FIG. 14.

### DETAILED DESCRIPTION OF THE INVENTION

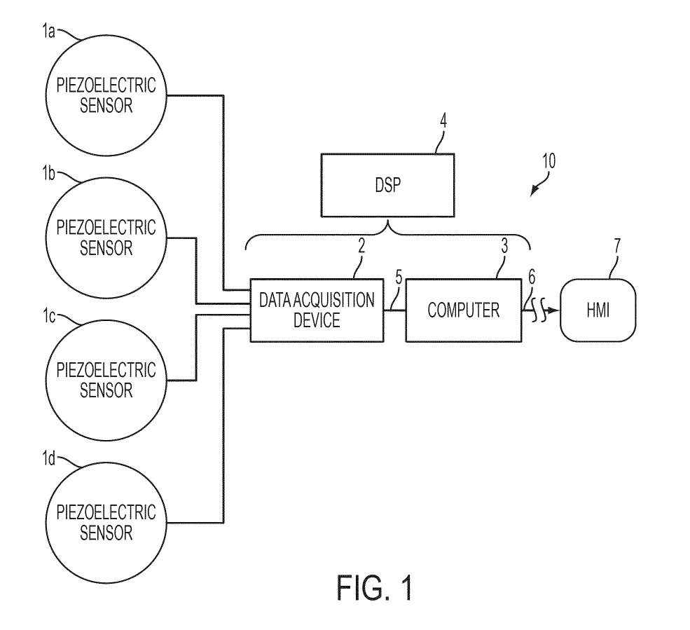

Turning now to FIG. 1, shown is a schematic block diagram of a system for the non-contact detection of physiological data. In this example, the physiological data of greatest interest are heart rate and respiration, the detection of which is performed by sensors 1a-1d, which in the embodiment shown are piezoelectric sensors. Force exerted on the sensors from pressure, acceleration, strain or other sources is converted to an electrical signal. In one embodiment, piezoelectric sensors 1a-1d use acceleration compensation elements in addition to pressure sensing elements to more accurately detect the physiological data in light of ambient noise. Although measurement of heart rate and respiration with the use of piezoelectric sensors is described, other physiological and biometric activity may be sensed and other types of non-contact sensors may be used.

In addition, the quantity of sensors used may be varied. In some embodiments, the signals output from the sensors are added together such that a specific number of sensors are not critical.

The outputs from the piezoelectric sensors 1a-1d are provided to a data acquisition device 2. The data acquisition device 2 serves as an analog-to-digital converter of the respective signals received from piezoelectric sensors 1a-1d. Via a single output 5 of the data acquisition device 2, the converted signals from piezoelectric sensors 1a-1d are then forwarded to a computer 3 for further processing. The computer 3 performs the stochastic resonance function (described below). After increasing a signal-to-noise ratio of the sensed physiological signals through the operation of the stochastic resonance function, the signals are further processed so as to differentiate the physiological signals from ambient noise and then measured for actual data values. The processed signals are then made available at an output 6 of the computer 3 for use by, for example, a human machine-interface (HMI) 7. The output 6 of the computer 3 may provide either a hard-wired or wireless communication connection to the HMI 7. The HMI 7 is just one form of a suitable HMI. A variety of different types of human-machine interfaces may be used.

Other varieties of the system shown in FIG. 1 are possible. Instead of the combination of the data acquisition device 2 and the computer 3, the functions of these two components may be combined into a single digital signal processor 4. In this case, the digital signal processor 4 performs the analog-to-digital conversion of the signals received from the piezoelectric sensors 1a-1d which in the previously described embodiment was performed separately by the data acquisition device 2.

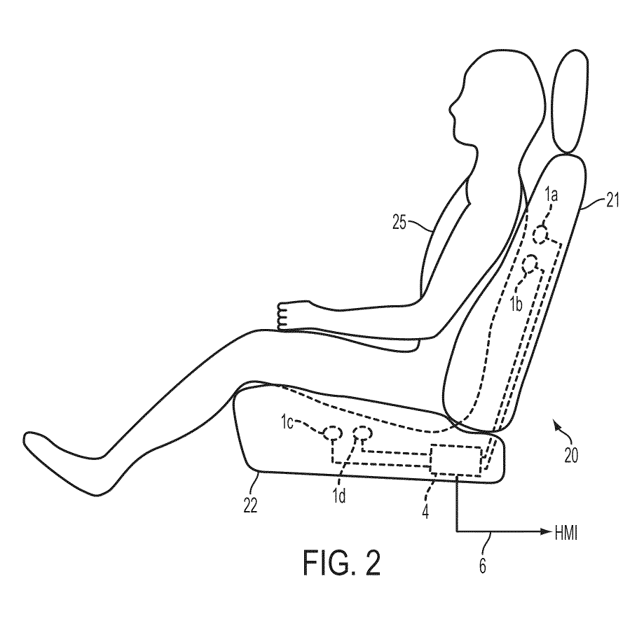

FIG. 2 shows a side view of one embodiment of the system of the present invention, indicated generally by 20, whereby an occupant 25 of a vehicle is sitting on a seat of the vehicle. Sensors 1a-1d are preferably positioned and secured within the seat so as to be in relatively close proximity to the body of the occupant 25 without being attached thereto. Preferably, two of the sensors 1a and 1b are positioned within a seat back 21 of the seat in close proximity to the rib cage of the occupant 25, while sensors 1c and 1d are positioned within a seat bottom 22 of the seat in close proximity to the thighs of the occupant 25. In addition to the desired physiological signals, sensors 1a-1d also detect, of course, any ambient noise in the general vicinity of the system 20.

The sensors 1a-1d are connected to the digital signal processor 4 (which is mounted within the seat bottom 22 of the vehicle seat in the embodiment shown). As is discussed, the digital signal processor 4 performs a stochastic resonance function on the signals received from sensors 1a-1d and subjects them to further processing so as to arrive at actual measured values of the desired physiological signals (for example, heart rate and respiration). The output 6 of the digital signal processor 4 is then made available to the HMI 7 for viewing and analysis.

In one example, the human-machine interface is a component of the associated vehicle and uses the physiological data to determine the overall physical condition of the occupant 25. In other implementations, the output 6 of the digital signal processor 4 is wirelessly connected for communication to a remotely-positioned human-machine interface.

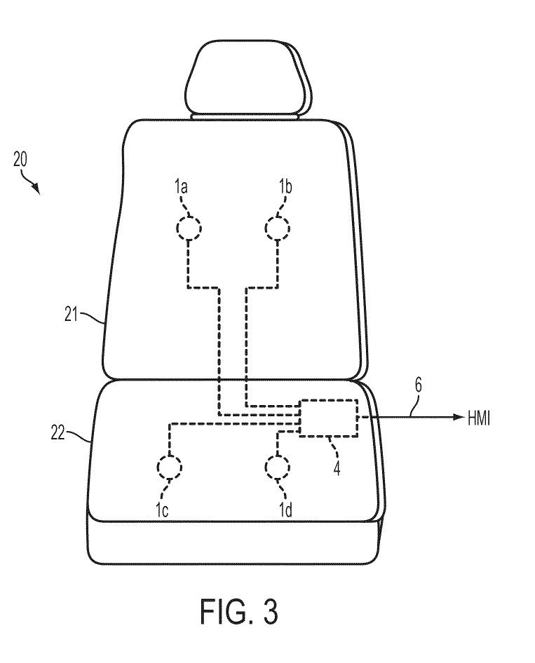

Referring now to FIG. 3, the system 20 is shown disposed in a vehicle seat as in FIG. 2. The sensors 1a and 1b are spaced apart from each other on either side of a center-line of the seat back 21 of the vehicle seat. The sensors 1c and 1d are similarly positioned but located in the seat bottom 22 along with the digital signal processor 4.

It should be understood that the system 20 for the non-contact detection of physiological data may be incorporated into a variety of seating arrangements upon which an occupant may be accommodated. Such may be a medical examination chair or other seat or furniture.

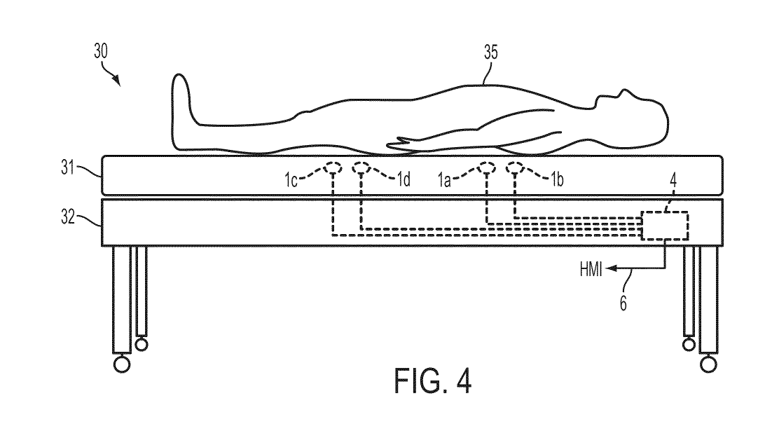

FIG. 4 is an alternative embodiment of the present invention whereby a system 30 for the non-contact detection of physiological data is incorporated into a hospital bed arrangement with an occupant disposed thereon. Specifically, sensors 1a-1d are preferably positioned within a mattress 31 of a hospital bed 32 so as to be in relatively close proximity to a surface of the body of an occupant 35. As shown, sensors 1a and 1b are preferably positioned in close proximity to the rib cage of the occupant 35 while sensors 1c and 1d are preferably positioned in close proximity to the thighs of the occupant 35. The sensors 1a-1d are connected to a digital signal processor 4 mounted within the hospital bed 32. The function of the system 30 shown in FIG. 4 will be similar to that as described in connection with the system 20 of FIG. 2.

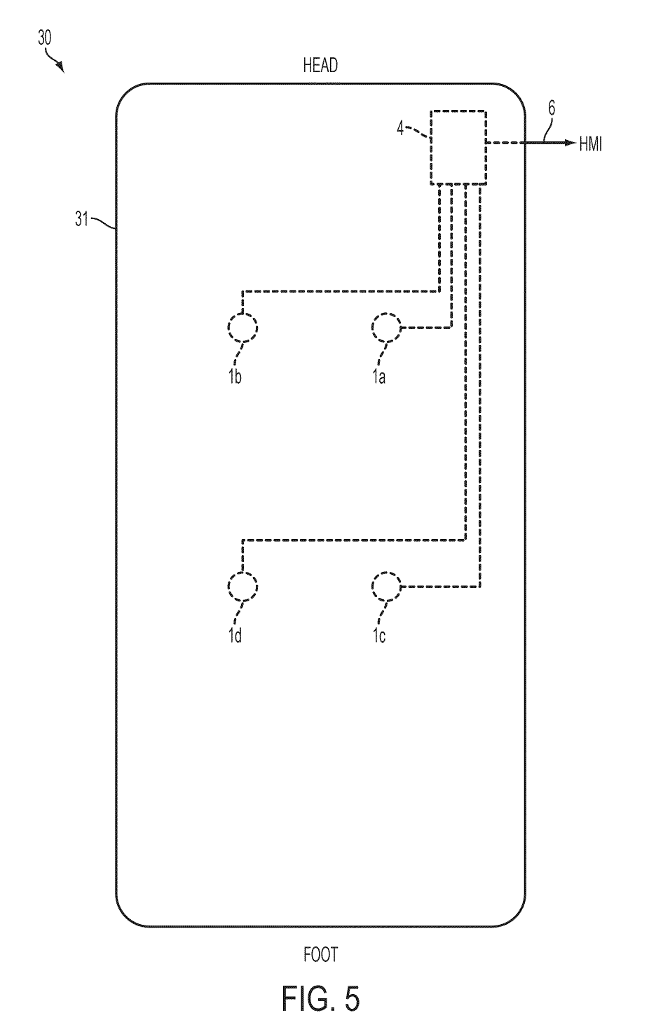

FIG. 5 shows a top view of the system 30 and mattress 31 from FIG. 4. The sensors 1a-1d are located in positions with respect to the head and foot of the mattress 31 as well as on either side of a center-line of the mattress 31. To clarify, the digital signal processor 4 is actually positioned and mounted within the hospital bed 32 shown in FIG. 4, the hospital bed 32 being located directly underneath mattress 31. Again, the actual number and positioning of physiological sensors may vary from that which is shown in FIG. 5 to accomplish the overall goals of the system 30.

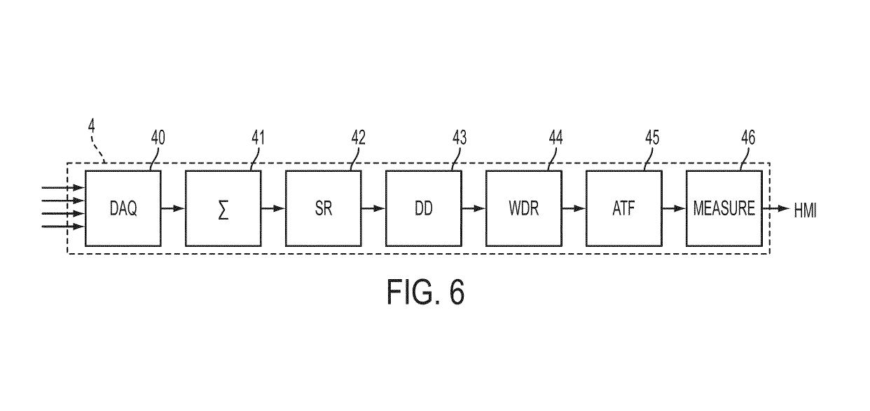

FIG. 6, illustrates components of the digital signal processor 4 as implemented in one embodiment. The components of the processor 4 may be implemented in hardware, software, or a combination of the same.

Physiological signals detected by the non-contact sensors are received by the digital signal processor 4 and inputted into a data acquisition module 40. The data acquisition module 40 performs an analog-to-digital conversion of the signals before further processing. The individual digital signals are then added together via the summation module 41 so as to further maximize peak amplitudes of the physiological signals with respect to ambient noise. Thereafter, the signals are processed by a stochastic resonance module 42 so as to further increase the signal-to-noise ratio of the physiological signals, as is discussed below.

Stochastic resonance is, in general, a resonance phenomenon that causes an increase of order in the response of the system due to a concurrence of noise and system non-linearities.

More particularly, stochastic resonance occurs in bi-stable systems when a relatively weak periodic force is applied together with a relatively large wide-band stochastic force (noise). System response is derived by the combination of these two forces, which both compete and cooperate to make the system switch between the two stable states. When a relatively weak periodic signal is so small so as to not make the system switch, the presence of ambient noise may cause a switch. It is believed that an optimal value of ambient noise exists that cooperatively works with the periodic signal to maximize the associated signal-to-noise ratio.

With respect to the embodiments described herein, we consider the stochastic resonance phenomenon in the context of a one-dimensional, bi-stable potential (or potential well). Such potential is weakly modulated by a non-linear, periodic signal—specifically, the physiological signals such as heart rate and respiration. The effect of an external input is to alternately raise and lower the level of the potential wells with respect to the effective area height between the wells. When ambient noise is added to such a system, a particle residing in one of the wells can move to the other through the stochastic activation. More particularly, in the presence of the relatively weak physiological signal modulation, the addition of ambient noise causes the transitions between the two states to become more coherent with the physiological signals.

In the presence of ambient noise, a conversion will occur whereby an increase in conversion rate is directly correlated to an increase in ambient noise. The conversion rate (R) may be represented by,

$$R = \frac{a}{π \sqrt{2}} exp [ - \frac{2 Δ V}{D} ]$$

where a is a predetermined parameter related to the amplitude of the desired signal and D is noise intensity.

It has been found that when the conversion rate (R) is equal to double the frequency of the periodical physiological signal, the signal-to-noise ratio of the physiological signal reaches its maximum level. When the signal-to-noise ratio is increased, obtaining or determining desired physiological data is made easier.

Experiments performed using sample ambient noise yielded a variety of experimental data. Such experiments involved specific embodiments of the invention whereby non-contact sensors were used to detect the physiological signals of an occupant of a car seat with ambient noise being present. The specific physiological signal in consideration was the occupant's heart rate or heart rate signal.

Two general parameters, a and b, relate to an amplitude, c, of the relatively weak, periodical heart rate signal. These parameters are related to noise intensity as follows,

$$\frac{a}{b} = {( \frac{{D}_{1}}{{D}_{0}} )}^{2}$$

where values of D₀ (noise intensity at time zero) and D₁ (noise intensity at time one) may be derived from experimental data. Moreover, it is not necessary to calculate a in this equation as a predetermined value (for example, 0.1) can be used for maximizing a signal-to-noise ratio. Accordingly, since values of a, D₀ and D₁ are all known, a value of b may be derived. Thereafter, the values for a and b may be substituted into the equation,

$$0 < c < {( \frac{4 {a}^{3}}{27 b} )}^{\frac{1}{2}}$$

to arrive at a maximum value of c. According to experimental data, the amplitude of the detected heart rate was 0.025V.

Both the theoretical and the functional aspects of stochastic resonance as described above are applicable to the methods and systems hereinafter described.

After the signals are processed in module 42, they are then forwarded to a data decimation module 43 which functions to take the signals from a relatively high sampling rate down to a lower user-defined sampling rate.

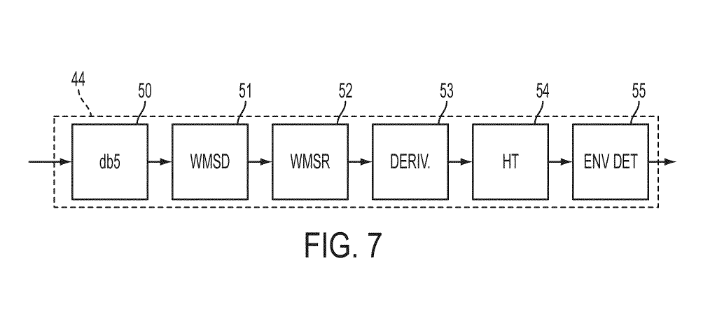

The next step in the processing of the physiological signals may be generally referred to as wavelet multi-scale decomposition (WMSD). In the illustrated embodiment, a db5 wavelet is used as part of WMSD and is employed by the wavelet decomposition and reconstruction (WDR) module 44. By way of the background, a wavelet is a mathematical function used to divide a given function into different scale components with a frequency range being assigned to each scale component. Each scale component may then be studied with a resolution that matches its scale. A wavelet transform is the representation of a function by wavelets. The wavelets are scaled and translated copies of a finite-length or fast-decaying oscillating waveform, known as a mother wavelet. One embodiment employs the use of Daubechies wavelet 5 as the mother wavelet. Wavelet transforms are advantageous for representing functions that have discontinuities and sharp peaks, and for accurately deconstructing and reconstructing finite, non-periodic signals. Specifics of the wavelet module 44 are further described in connection with FIG. 7.

After the processing and reconstruction of the physiological signals in the WDR module 44, an adaptive threshold is applied to the periodic physiological signal waveforms via the adaptive threshold function module 45. The physiological signals detected by the non-contact sensors vary in intensity depending upon the subject's movement with respect to the sensors. As a consequence, identifying the peaks corresponding to the desired physiological signals was found to be difficult in a number of circumstances. The adaptive threshold function module 45 sets a threshold whereby waveform peaks above the threshold are identified (or classified as) as the physiological signals and waveform peaks below the threshold are classified as ambient noise. The threshold is “adaptive” in that it is automatically adjusted depending upon the changing nature of the signal over time. After being identified, peaks of the physiological signals are converted from a point scale back to the frequency domain so as to result in actual data values being output by the signal measurement module 46. The resulting data from the measurement module is then output by the digital signal processor 4.

FIG. 7 shows an internal structure of the basic parts of the WDR module 44 shown in FIG. 6. A wavelet decomposition and reconstruction is a way to decompose a signal into multiple different scales frequency and obtain corresponding detailed and approximate expressions of data. Wavelets are a mathematical tool they can be used to extract information from many different kinds of data, including—but certainly not limited to—audio signals and images. Sets of wavelets are generally needed to analyze data fully. The db5 wavelet module 50 selected is the first to process the signals which are received from the data decimation module 43 of FIG. 6. Pursuant to db5 wavelet module 50, a complicated data set can be decomposed into a finite and often small number of fundamental components. That is, after decimation there still may be many signals present—many sine waves added together. Performing a frequency analysis of these sine waves is difficult when the frequencies of the sine waves are very close together or overlap. Without having to actually perform a specific frequency analysis, db5 wavelet module 50 can break down the signals, or “decompose” the signals, into a finite number of fundamental components. The fundamental components are actually a collection of multiple frequency scales components. Once db5 is determined, it is natural to carry out the following WMSD method. As discussed earlier, an embodiment of the present invention uses Daubechies wavelets as a basis for analyzing the detected signals. Specifically, a db5 component is used to decompose the original signals into five scales from high frequency to low frequency components.

Once decomposed, the signals are then forwarded to a wavelet multi-scale decomposition (WMSD) module 51, which tests the signals. The WMSD module 51 looks for a candidate signal that it decomposes and gives corresponding expressions of data. Then it is forwarded to the wavelet multi-scales reconstruction module (WMSR) 52. The (WMSR) module 52 reconstructs a signal waveform across the entire frequency domain which had previously been deconstructed via the WMSD module 51.

At this point, it can be determined which of the fundamental components decomposed by the WMSD 51 most likely represents the specific physiological signals of interest. Then, a first order derivative is calculated at the custom derivative function module 53 in order to strengthen a peak value of the identified signals and to see if the major frequency components are present for the desired physiological signals. For example, if the custom derivative function module 53 sees a frequency of 5 Hz (which, for a heart rate, corresponds to 300 beats per minute) it would determine that this signal does not, in fact, correspond to a heart rate (because the signal is far outside the range of a normal or expected heart rate). If the criteria are satisfied, the signals are forwarded to the Hilbert Transform module 54. The Hilbert Transform module 54 is used to derive an analytic representation of the physiological signals. Performing a Hilbert Transform allows for the effective and subsequent performance of the envelope detector module 55. As shown in FIG. 7, the envelope detector module 55 receives a signal from the Hilbert Transform module 54 whereby the envelope detector module 55 extracts and forms the envelope of original signals peaks via the method of promoting big peaks and compressing small peaks. Following the processes in the WDR module 44, the resulting output signal is forwarded to the adaptive threshold function module 45 of FIG. 6.

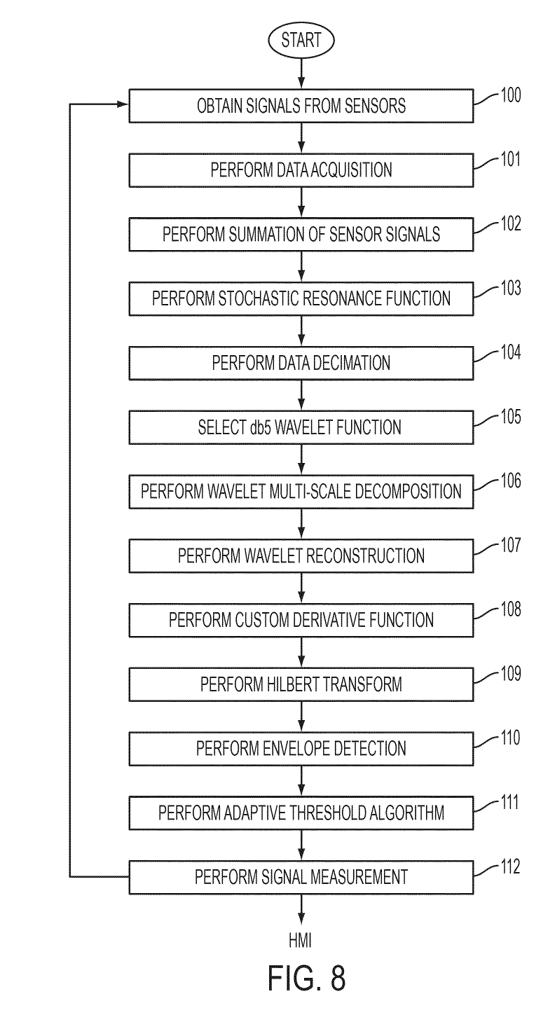

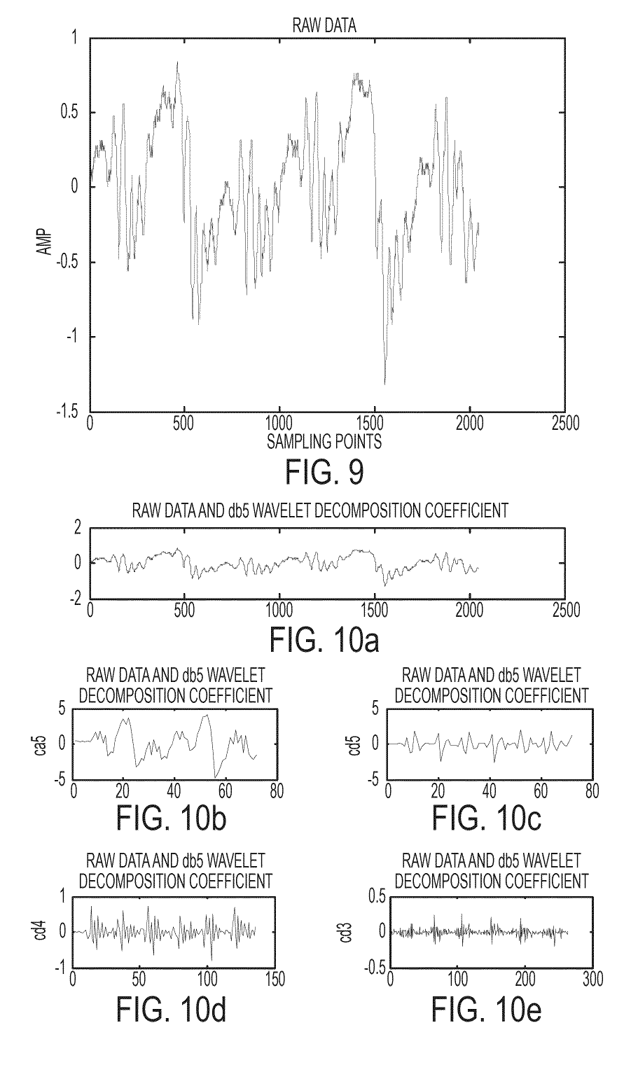

An overview of an embodiment will now be described in connection with the flow chart shown in FIG. 8. At step 100, signals are obtained from the non-contact sensors. As noted, the signals may include both the desired physiological signals and ambient noise. A data acquisition step 101 is then performed on the signals to convert them into a digital form. A graphical waveform representation of an output signal after the data acquisition step 101 may be seen in FIG. 9. Thereafter, a summation of the sensor signals is performed at step 102 whereby the signals are added together. The signals are then processed in a stochastic resonance function at step 103. The use of stochastic resonance in combination with the non-contact sensors has proven to be particularly beneficial in accurately identifying and measuring weak, periodic physiological signals in light of even significant ambient noise.

After processing in the stochastic resonance function, a data decimation step 104 is performed to take the signals from a high sampling rate down to a lower, user-defined sampling rate. At this point, the wavelet multi-scale decomposition function begins.

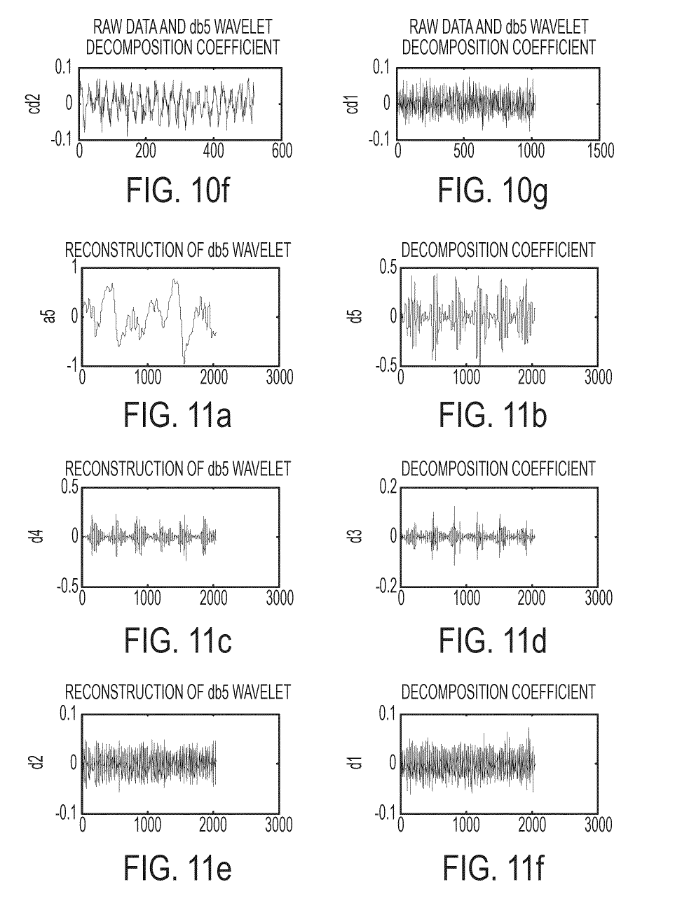

As previously described, the db5 wavelet is employed in module 105 to analyze example signals into 5 scales from high frequency to low frequency components through the module 106, resulting in the graphical waveforms shown in FIGS. 10a-10g.

Reconstruction of the db5 wavelet, after decomposition performed in step 106, is performed at the wavelet reconstruction step 107. The resulting signal output after the wavelet reconstruction step 107 is shown in the graphical waveforms of FIGS. 11a-11f.

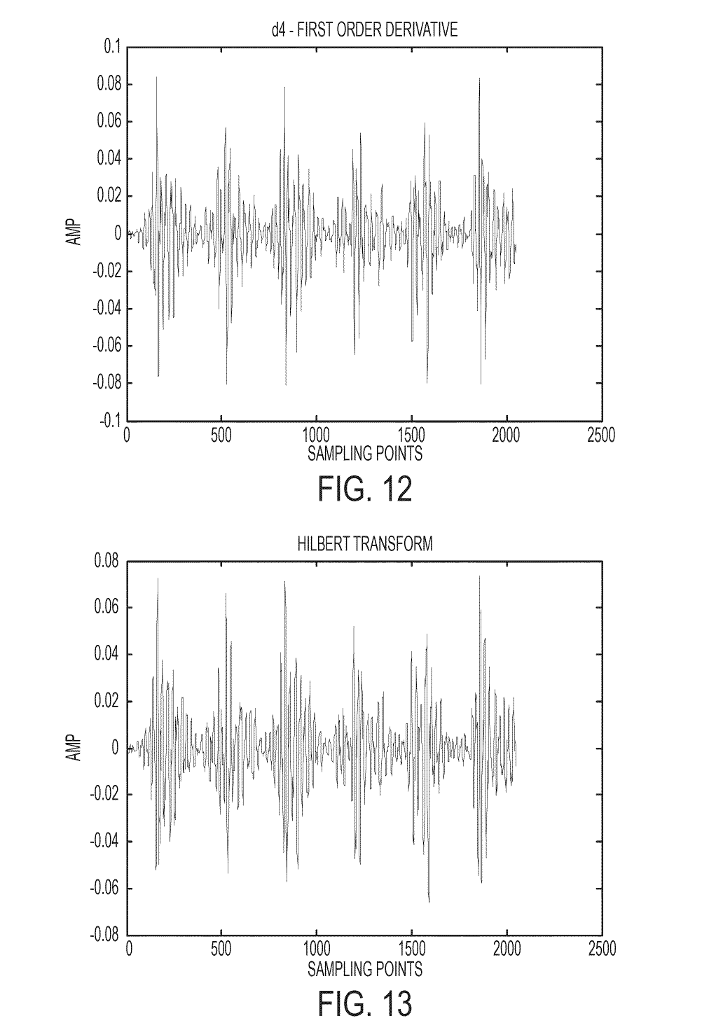

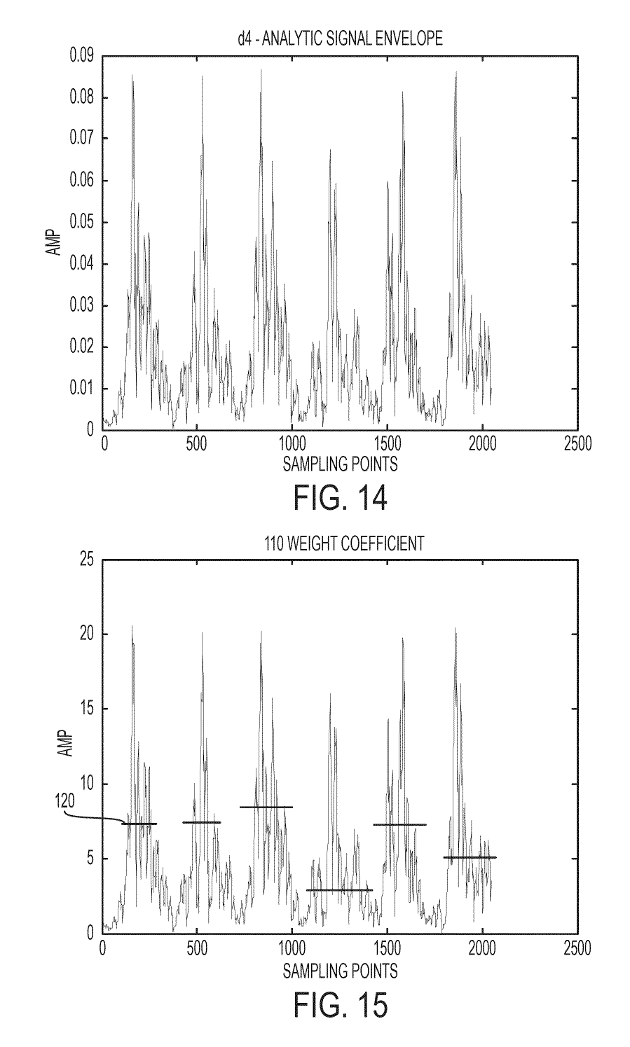

As discussed earlier, based on empirical evidence, one of the fundamental components as shown in FIGS. 11a-11f will have characteristics most closely associated with the physiological signals being sensed. In the present case, the likely candidate is the d4 component shown in FIG. 11c. Accordingly, a first order derivative of such signal is taken at the step of performing a custom derivative function 108. The resulting waveform with a strengthened peak value is graphically shown in FIG. 12. A Hilbert Transform is thereafter performed at step 109 on the transmitted signal with a resulting graphical waveform as shown in FIG. 13. Next, an envelope detector step 110 is performed whereby the negative frequencies of the waveform received are eliminated and peak values of the sensed signals are stressed as shown in FIG. 14.

FIG. 15 shows the application of an adaptive threshold 120 to a resultant waveform as per the adaptive threshold function step 111. As can be seen, the adaptive threshold 120 adjusts its level over time. Finally, the data obtained may be processed and converted into actual measurement data of the desired physiological signals as per the signal measurement step 112. The resulting information then may be made available to a human-machine interface. Additional information may be processed by repeating the process and returning to step 100.

While the embodiments have been described with reference to specific examples, those of skill in the art will recognize that changes may be made thereto without departing from the spirit and scope of the present invention as set forth in the appended claims. For example, the invention may be embodied in a variety of horizontal and substantially planar surface arrangements. Various beds, tables, chairs, or other devices could accept such a system like the system 30 to monitor desired physiological signals of a living being, such as a human or animal.

1. A system for sensing and measuring physiological signals of a living being, the system comprising:
a plurality of non-contact sensors disposed to sense the physiological signals along with ambient noise; and
a processor connected to the plurality of the sensors for receiving the physiological signals and the ambient noise, wherein the processor includes;
a stochastic resonance module for performing a stochastic resonance function on the physiological signals and the ambient noise to increase a signal-to-noise ratio of the physiological signals;
an identifying module for distinguishing the physiological signals from the ambient noise; and
a measuring module for measuring values associated with the physiological signals.

2. A system as claimed in claim 1, wherein the physiological signals are at least one of a heart rate and respiration.

3. A system as claimed in claim 1, wherein the plurality of sensors are piezoelectric pressure sensors.

4. A system as claimed in claim 1, wherein the plurality of sensors are disposed within a seat.

5. A system as claimed in claim 4, wherein the seating apparatus is at least one of a seat of a vehicle and a medical examination chair.

6. A system as claimed in claim 1, wherein the plurality of sensors are disposed within a bed.

7. A system as claimed in claim 1, further comprising a data acquisition device, wherein the data acquisition device receives the physiological signals and the ambient noise from the plurality of sensors, performs an analog-to-digital conversion of the physiological signals and the ambient noise, and outputs the digital physiological signals and ambient noise to the processor.

8. A system as claimed in claim 1, wherein the apparatus further includes a summation module for adding the physiological signals together prior to being input into the stochastic resonance module.

9. A system as claimed in claim 1, wherein the apparatus further includes a data decimation module for taking the physiological signals and the ambient noise from a high sampling rate down to a lower sampling rate.

10. A system as claimed in claim 1, wherein the identifying module includes a wavelet multi-scale decomposition function.

11. A system as claimed in claim 10, wherein a db5 wavelet is used as a mother wavelet of the wavelet multi-scale decomposition function.

12. A system as claimed in claim 1, wherein the identifying module includes an envelope detector for eliminating negative frequencies of the physiological signals and the ambient noise.

13. A system as claimed in claim 1, wherein the identifying module includes an adaptive threshold function for incorporating an adaptive threshold between the physiological signals and the ambient noise.

14. A method for sensing and measuring physiological signals of a living being, the method comprising:
sensing the physiological signals along with ambient noise using a plurality of sensors disposed in non-contact proximity to the living being;
performing a stochastic resonance function on the physiological signals and the ambient noise to increase a signal-to-noise ratio of the physiological signals;
distinguishing the physiological signals from the ambient noise after the signal-to-noise ratio has been increased; and
measuring values of the physiological signals that have been distinguished from the ambient noise.

15. A method as claimed in claim 14, wherein the physiological signals are at least one of a heart rate and a respiration rate.

16. A method as claimed in claim 14, wherein the plurality of sensors are piezoelectric pressure sensors.

17. A method as claimed in claim 14, wherein the plurality of sensors are disposed within a seat.

18. A method as claimed in claim 17, wherein the seat is at least one of a seat of a vehicle and a medical examination chair.

19. A method as claimed in claim 14, wherein the plurality of sensors are disposed within a bed.

20. A method as claimed in claim 14, further comprising performing an analog-to-digital conversion of the physiological signals and the ambient noise prior to performing the stochastic resonance function.

21. A method as claimed in claim 14, further comprising adding the physiological signals together prior to performing the stochastic resonance function.

22. A method as claimed in claim 14, further comprising performing a data decimation function to take the physiological signals and the ambient noise from a high sampling rate down to a lower sampling rate.

23. A method as claimed in claim 14, wherein distinguishing the physiological signals from the ambient noise includes performing a wavelet multi-scale decomposition function.

24. A method as claimed in claim 23, wherein a db5 wavelet is used as a mother wavelet of the wavelet multi-scale decomposition function.

25. A method as claimed in claim 14, wherein distinguishing the physiological signals from the ambient noise includes performing an envelope detector function for eliminating negative frequencies of the physiological signals and the ambient noise.

26. A method as claimed in claim 14, wherein distinguishing the physiological signals from the ambient noise includes performing an adaptive threshold function for incorporating an adaptive threshold between the physiological signals and the ambient noise.

27. An apparatus for processing physiological signals of a living being, the apparatus comprising:
an input for receiving the physiological signals along with ambient noise that have been sensed by a plurality of sensors disposed in non-contact proximity to the living being;
a stochastic resonance module for performing a stochastic resonance function on the physiological signals and the ambient noise to increase a signal-to-noise ratio of the physiological signals;
an identifying module for distinguishing the physiological signals from the ambient noise; and
a measuring module for measuring values associated with the physiological signals.
